(19) United States
(12) Patent Application Publication
(10) Pub. No.: US 2026/0198856 A1
(43) Pub. Date: Jul. 16, 2026

(54) "SMART" BAG AND ITEM OF CLOTHING COMPRISING SUCH "SMART" BAG

(71) Applicants: Techinnova SPA, Milano (IT); MTM S.R.L., Milano (IT)

(72) Inventors: Riccardo ROGGERI, Saltrio (IT); Sayed Amir PARSAFAR, Sesto San Giovanni (IT)

(21) Appl. No.: 19/134,676

(22) PCT Filed: Dec. 5, 2023
(86) PCT No.: PCT/IB2023/062233

§ 371 Date: May 30, 2025

(30) Foreign Application Priority Data
Dec. 6, 2022 (IT) 102022000025131

(51) Int. Cl. A61B 5/00; A61B 5/0205

(52) U.S. Cl. CPC A61B 5/6804; A61B 5/0205; A61B 2560/0242; A61B 2560/0462

(57) ABSTRACT

“Smart” bag (1) configured to be connected to an item of clothing (4), said bag (1) comprising:—one hardware components pouch (2), having a front face (20) and a rear face opposite to the front face (20), between the front face (20) and the rear face A1 at least one pocket (21) being delimited, which pocket is in communication with the outside of pouch (2) through one opening and, inside the pocket (21) being housed a plurality of detectors (3), each detector (3) being configured to detect one or more environmental parameters about the environment around the bag (1) or one or more physiological parameters of a user, when the user is wearing an item of clothing (4) connected to the bag (1), in reply to a respective activation signal sa(t) received in input, and output a corresponding detection signal sₛ(t);—a data control and processing unit (5), operatively connected to the plurality of detectors (3) and configured to send said activation signal sa(t) to each one of said detectors (3) and receive, in reply, the corresponding detection signal sₛ(t). The bag (1) further comprising a plurality of connection elements (7), configured for removably connecting the hardware components pouch (2) to the item of clothing (4).

The present invention concerns the “smart” clothing sector, i.e. the sector of items of clothing equipped with electrical/electronic devices capable of collecting, storing and exchanging information from and towards the surrounding environment, for example information relating heart rate, breathing rate, body temperature, etc. of those who wear them while carrying out a physical activity or at rest, or capable of collecting, storing and exchanging information relating to the environment in which they are, such as environmental temperature, atmospheric humidity, position, etc.

In recent years, with the development of new technologies and changes in lifestyle, there has been an increase in the use of “smart” clothing. Some examples of such items are described in US2012/146797 A1, EP2505090 A2, US2007/032981 A1, US2015148619 A1, US2014135593 A1, GB2589360 A, and WO19068902 A1.

Although this sector shows constantly growing numbers, there are currently various limitations that slow down its development on the market.

Among the main limitations, for example, we find the fact that a traditional “smart” item of clothing is designed to perform a limited number of functions, for example only measuring heart rate and body temperature. Another drawback concerns the limited availability of sizes, which often make it unusable for overweight people or people with a too thin build. A further limitation is represented by its high costs. Yet another limit is represented by the discomfort felt by the person wearing it, as an item of clothing of this type is usually made with non-breathable materials and generally has electrical/electronic components designed to come into direct contact with the user's body, with the risk that, over time, they cause discomfort or skin irritation due to rubbing. Finally, a further important limitation is represented by the impossibility of washing a traditional “smart” item of clothing without running the slightest risk of damaging the electrical/electronic components incorporated therein.

There is therefore a need to improve the state of the art in the “smart” clothing sector and the object of the present invention is to allow the aforementioned limitations to be resolved or at least mitigated.

The specific object of the present invention is a “smart” bag configured to be connected to an item of clothing, said bag comprising:
one hardware components pouch, having a front face and a rear face opposite to the front face, between the front face and the rear face at least one pocket being delimited, which pocket is in communication with the outside of pouch through one opening and, inside the pocket being housed:
   a plurality of detectors, each detector being configured to detect one or more environmental parameters about the environment around the bag or one or more physiological parameters of a user, when the user is wearing an item of clothing connected to said bag, in reply to a respective activation signal sₐ(t) received in input, and output a corresponding detection signal sₛ(t);
   a data control and processing unit, operatively connected to the plurality of detectors and configured to send said activation signal sₐ(t) to each one of said detectors and receive, in reply, the corresponding detection signal sₛ(t); and
a plurality of connection elements, configured for removably connecting the hardware components pouch to said item of clothing;
   wherein
   each connection element of the plurality of connection elements comprises:
at least one first portion, configured for being fixed on the same face of the hardware components pouch, optionally in a permanent or removable way; and
at least one second portion, configured, to be removably engaged with the respective first portion, on one side, and to be applied to the item of clothing, on the other side.

According to a further aspect of the invention, the second portion can be configured to be applied to the item of clothing, optionally permanently or removably, at one inner face of said item of clothing facing the body of the user, when the user is wearing said item of clothing.

According to an additional aspect of the invention, the second portion can be configured to be applied to the item of clothing, optionally in a permanent or removable way, at one outer face of said item of clothing, facing the environment around the user, when the user is wearing said item of clothing.

According to another aspect of the invention, the removable engagement between the first portion and the second portion, can be a mechanical and/or magnetic engagement.

According to a further aspect of the invention, the first portion and the second portion at least one magnet is housed, optionally four magnets, when the engagement between the first portion and the second portion is of magnetic type.

According to an additional aspect of the invention, at least one connection element can also comprise a reference third portion, the reference third portion being configured to be directly engaged with the item of clothing, when the second portion of the respective connection element is applied to the item of clothing at the inner face thereof, or configured to be applied to the item of clothing directly engaged with the second portion of the connection element, when the second portion of the connection element is applied to the item of clothing, at the outer face of said item of clothing, the outer face of the reference third portion optionally having some raised areas, forming a number and/or a sign in braille writing, which identifies each connection element.

According to another aspect of the invention, the bag can comprise a plurality of emitting devices housed inside the pouch, each emitting device being arranged at a first portion of a respective connection element, and being operatively connected to the data control and processing unit and being configured for:
being activated, in reply to a corresponding control signal s_{c}(t) sent by the data control and processing unit, and
emitting a corresponding emission signal sₑ(t).

According to a further aspect of the invention, the bag can comprise a plurality of switching devices housed inside the pocket, each switching device being arranged at a first portion of a respective connection element, and being operatively connected to the data control and processing unit and configured for being manually operated by a user, through a pressure exerted at least on the respective second portion, and emitting in reply a corresponding switching signal s_{sw}(t) toward the data control and processing unit.

According to an additional aspect of the invention, the hardware components pouch can be made of a breathable fabric and/or each detector, each emitting device and each switching device is operatively connected to the data control and processing unit through a conductive yarn.

According to another aspect of the invention, the bag can comprise at least one I/O interface, housed inside the pouch and operatively connected to the data control and processing unit, and configured for allowing one output signal sₒᵤₜ(t) transmission toward the outside to a remote device, said signal being outputted by the data control and processing unit, and for allowing receiving in input and transmitting to the data control and processing unit one or more input signals sᵢₙ(t), transmitted by the remote device, the remote device optionally being a portable phone, tablet, pc, a remote server or other suitable processing unit.

It is also the subject of the present invention an item of clothing connected to a smart bag according to any previous statement.

A further object of the present invention is a method of use of a bag as described above, when the bag is applied to an item of clothing, the method comprising the following operational steps:
A. through the data control and processing unit, sending respective control signals s_{c}(t) to a plurality of emitting devices, according to a predefined delivery order, so that each emitting device, reached by the respective control signal s_{c}(t) emits one emission signal sₑ(t);
B. after a preset time interval, detecting through the data control and processing unit any switching signal s_{sw}(t) emitted by respective switching devices, in reply to a pressure exerted by the user on the respective connection element, and storing the order of arrival of said switching signals s_{sw}(t);
C. through the data control and processing unit (5):
comparing the predefined delivery order of the control signals s_{c}(t) with the order of arrival of any received switching signals s_{sw}(t), and
storing the result of said comparison.

According to another aspect of the invention, the method can comprise a step D of emitting, by the data control and processing unit toward I/O interface, one output signal sₒᵤₜ(t) based on the result of said comparison carried out at step C, for forwarding it by said I/O interface to the remote device and its subsequent processing.

According to a further aspect of the invention, the method can comprise, before step A, also one step A₀ of emitting a control signal s_{c}(t) toward a sound emitting device, if provided, that causes the emission of a predefined sound emission signal sₑ(t), to acoustically warn about the start of transmission of control signals s_{c}(t) according to the predefined delivery order, by the data control and processing unit (5),

and/or comprising after step C, or D is provided, one step E of emitting a control signal s_{c}(t) toward the sound emitting device, if provided, that causes the emission of a sound emission signal sₑ(t), the sound of which depends of the result of the comparison carried out at step C, to acoustically warn the user of the match or not match of the predefined delivery order of control signals s_{c}(t) with the order of arrival of the possibly received switching signals s_{sw}(t).

The present invention will now be described, by way of illustration but not limitation, according to preferred embodiments thereof, with particular reference to the attached figures,

wherein:

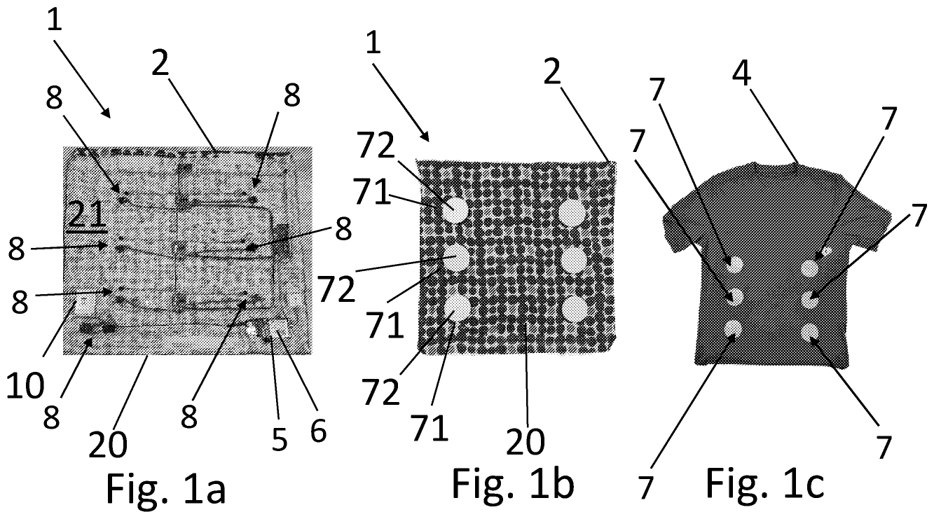

FIG. 1a schematically illustrates the inside of a hardware components pouch of the “smart” bag according to the present invention with its main electrical/electronic components;
FIG. 1b shows the outside of the hardware components pouch of FIG. 1a, on which first portions of connection elements to an item of clothing are applied;

FIG. 1c is a top view of an item of clothing connected to the smart bag of the present invention, on which second portions of connection elements to the hardware components pouch of FIG. 1b are applied;

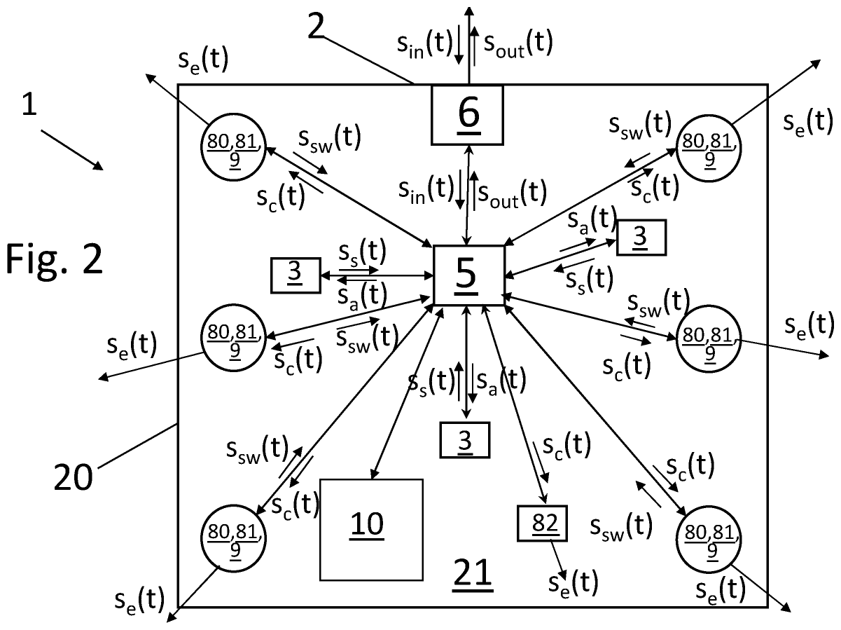

FIG. 2 shows a non-scale schematic representation of the distribution and mutual connection of the main electrical/electronic components in the hardware component pouch of the “smart” bag of the present invention;

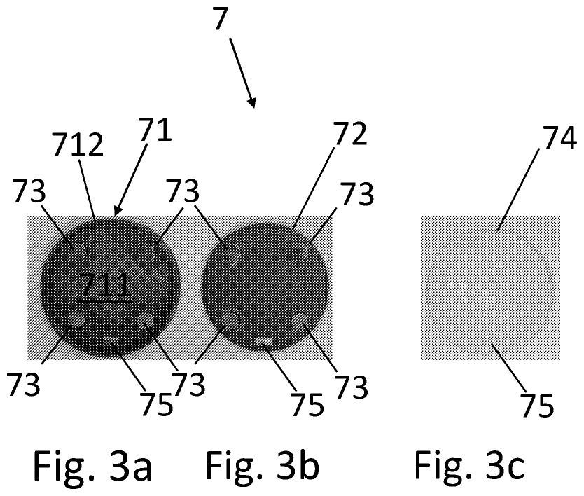

FIG. 3a is a representation of an embodiment of a first portion of a connection element of the “smart” bag according to the present invention;

FIG. 3b shows a representation of an embodiment of a second portion of a “smart” bag connection element, configured to directly or indirectly engage with the first portion of FIG. 3a;

FIG. 3c illustrates a representation of an embodiment of a reference third portion of a “smart” bag connection element, configured to directly or indirectly engage or with the second portion of FIG. 3b;

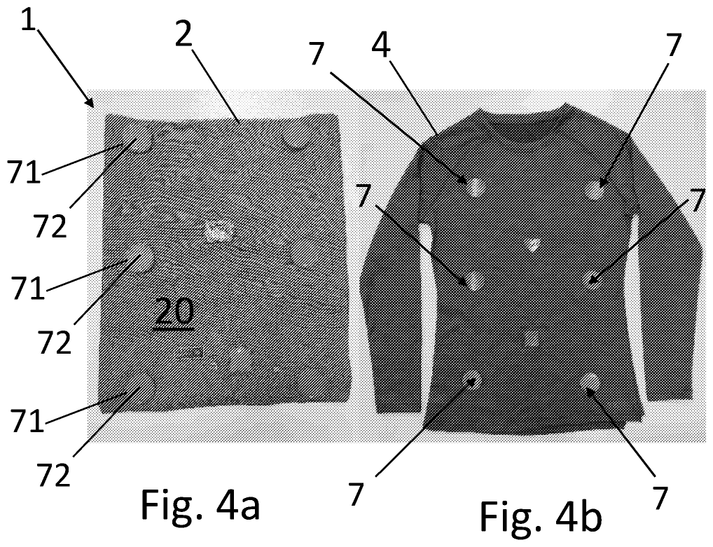

FIG. 4a illustrates a view from the outside of another embodiment of a smart bag according to the present invention;

FIG. 4b shows an item of clothing connected to the smart bag in FIG. 4a; and

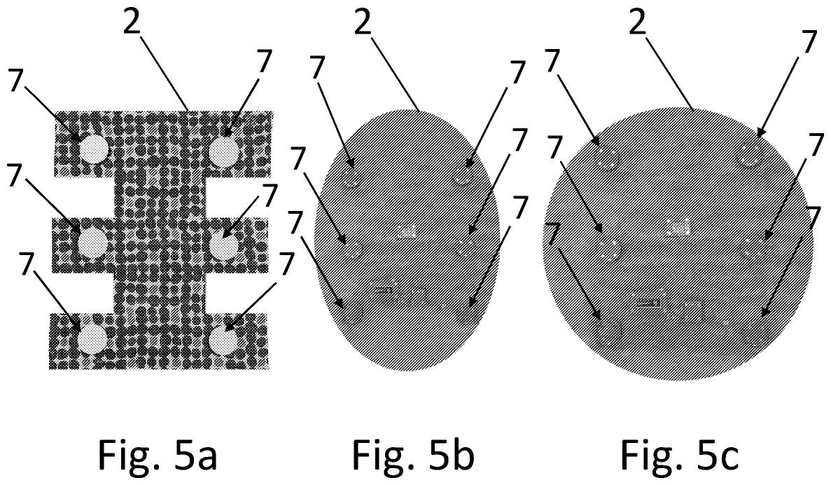

FIGS. 5a through 5c are representations of some alternative shapes of the hardware component pouch of the “smart” bag of the present invention.

With particular reference to FIGS. 1a, 1b, 2 and 4a and from 5a to 5c, it can be observed how a “smart” bag according to the present invention which, as will be seen, is configured to be connected to an item of clothing, is generally indicated with the reference number 1 and comprises at least one hardware components pouch 2, advantageously made of a breathable fabric, for example cotton or microfiber or other fabric of any suitable type. Such pouch 2 has a front face, illustrated in the drawings with reference number 20, and a rear face opposite to the front face 20, and delimits, between the front face 20 and the rear face at least one pocket 21 for housing hardware components, the pocket 21 being communicating with the outside of the pouch by means of an opening (not shown in the figure).

According to a preferred embodiment illustrated in FIGS. 1a, 1b, 2 and 4a, the hardware components pouch 2 has a substantially rectangular shape, with dimensions between 25 cm×20 cm and 42 cm×40 cm, which substantially correspond to the thoracic surface of a person of small or medium build, even if the person skilled in the art will have no difficulty in understanding how the shape of the hardware components pouch 2 can also be different, for example polygonal or oval or circular as shown in FIGS. 5a to 5c with maximum dimensions such as those indicated above or different, provided that it adequately performs its function of housing the hardware components which will be better illustrated below.

In the pocket 21 of the hardware component pouch 2 of the “smart” bag 1 of the invention a plurality of detectors 3 is housed, each detector 3 being configured for:
detecting, in reply to a respective activation signal (sₐ(t)) received as input, one or more environmental parameters regarding the environment surrounding the bag 1, or one or more physiological parameters of a user, when the user is wearing an item of clothing 4 connected to that smart bag 1, and
outputting a corresponding detection signal (sₛ(t)).

According to a preferred embodiment of the present invention, such detectors 3 are selected from: environmental temperature detectors, environmental humidity detectors, environmental pressure and altitude detectors or heart rate detectors, respiratory rate detectors, heart rate detectors, blood oxygenation detectors, body temperature detectors or even an inertial unit, for example comprising a gyroscope and a 3-axis accelerometer, to detect the position and speed of the “smart” bag with respect to the ground and, accordingly, the position and speed of the user wearing it. The detectors 3 are housed in the hardware components pouch 2 in a suitable location depending on the type of detection to be performed. For example, a body temperature detector 3 can be mounted in the pouch in any position, as long as it can accurately measure the body temperature of a user, when the user is wearing an item of clothing 4 connected to the “smart” bag 1 of the invention. Specifically, according to a preferred embodiment of the invention, the body temperature detector 3 is an IR sensor that measures the temperature through the emission of infrared waves, and therefore it can be housed in the pocket 21 of the pouch 2 in any position, as long as it maintains an adequate distance between the emission point of the IR waves used for the measurement and the user's body, optionally less than or equal to 10 mm. In this case, in the pouch 2 there is a through opening (not shown in the drawings) at the rear face which, in use, is facing the user's body, so that the IR waves emitted by the temperature detector 3 directly hit the body of the user. Again, by way of example, a heart rate detector 3, optionally a pulse oximeter, will have to be housed in the pouch 2 in a suitable position, such that, when a user is wearing an item of clothing 4 connected to the “smart” bag 1 of the invention, the detector is able to correctly detect the heart rate. According to a preferred embodiment of the invention, it is not necessary for the heart rate detector 3 to be positioned close to the heart of a user, since in order to measure the heart rate the user must exert a certain pressure with a finger, optionally constant over time, within a pre-established pressure range, on the detector itself. Also in this case, in the pouch 2 a through opening is obtained (not shown in the drawings) at the front face 20 thereof which, in use, is facing outwards, so that the detector can be easily reached with a finger by the user, to carry out the measurement. Advantageously, in an item of clothing 4 connected to the “smart” bag 1 of the invention, a through opening can be obtained at the heart rate detector 3, so that this is directly accessible from the outside of the item of clothing 4.

The “smart” bag 1 of the present invention also includes, a data control and processing unit 5 housed in the hardware components pouch 2, which is operatively connected to the plurality of detectors 3 and configured to send to ach one of such detectors 3 the aforementioned activation signal sₐ(t) and receive in response the corresponding detection signal sₛ(t).

The data control and processing unit 5 is further configured to process the aforementioned received detection signals sₛ(t), and provide a corresponding output signal sₒᵤₜ(t).

According to a preferred embodiment of the invention, the hardware components pouch 2 can comprise at least one I/O interface 6, operatively connected to the data control and processing unit 5, for example a USB port or a transceiver module of any suitable type, configured to allow the transmission of such output signal sₒᵤₜ(t) externally, for example to a remote device (not shown in the drawings), and to allow receiving as input and transmitting one or more input signals sᵢₙ(t) transmitted by the remote device to the data control and processing unit 5, the remote device being for example a portable telephone, a tablet, a PC, a remote server or any other suitable data processing unit.

Advantageously, the “smart” bag 1 of the present invention also comprises a plurality of connection elements 7 (see FIGS. 1b, 1c, 3a-3c, 4a and 4b and from 5a to 5c), made for example of a photopolymer resin, optionally PLA, and configured to removably connect the hardware components pouch 2 to an item of clothing 4, for example a shirt or tank top or trousers.

More particularly, see FIGS. 3a-3c, each connection element 7 of the plurality of connection elements comprises:
at least one first portion 71, configured to be fixed to the hardware components pouch 2, for example to its front face 20, optionally permanently, for example by means of a suitable glue, or in a removable way, for example by velcro, buttons or screws; and
at least a second portion 72, configured, on one side, to be removably connected with the respective first portion 71, and on the other side, configured to be applied to the item of clothing 4.

According to a first variant of the “smart” bag 1 of the present invention, the second portion 72 of a connection element 7 is configured to be fixed to the item of clothing 4, optionally permanently, for example glued or sewn to such item of clothing 4, or in a removable way, for example through velcro, buttons or screws, at an internal face of said item of clothing 4, i.e. a face which is facing the body of a user, when the user is wearing this item of clothing. In this case, when the “smart” bag 1 is connected to an item of clothing 4, the first portion 71 and the second portion 72 of that connection element 7 are designed to come into direct contact with each other and to create a removable engagement between the hardware components pouch 2 and item of clothing 4.

According to a second variant of the “smart” bag 1 of the present invention, the second portion 72 of a connection element 7 is configured to be applied to the item of clothing 4, optionally permanently, for example by means of a suitable glue, or in a removable way, for example by means of velcro, buttons or screws, at an external face of such item of clothing 4, i.e. a face which is turned towards the environment surrounding a user, when the user is wearing that item of clothing 4. In this second case, when the “smart” bag 1 is connected to an item of clothing 4, the first portion 71 and the second portion 72 of this connection element 7 are designed to mutually engage in a removable manner with each other, with the interposition of the item of clothing 4, which therefore remains sandwiched between them and with them creates a removable engagement between the hardware components pouch 2 and the item of clothing 4.

According to a particularly advantageous aspect of the invention, in both cases described above, the removable engagement which is created in use between the first portion 71 and the second portion 72 of each connection element 7 is a mechanical and/or or magnetic engagement.

In fact, as represented in FIGS. 3a and 3b, the first portion 71 and the second portion 72 are shaped so that one can be inserted into the other, substantially obtaining a form fitting. The first portion 71 comprises a circular base 711 and a continuous side wall 712 extending from the circular base 711 and delimiting therewith a housing seat. The second portion 72 is shaped as a disc, with a diameter slightly smaller than that of the circular base 711 of the first portion 71 and can therefore be inserted substantially to size into the housing seat of the latter. The first portion 71 and the second portion 72 of a connection element 7 can also have a different shape, for example with a polygonal plan, as long as they allow to substantially obtain a mutual form fitting of a removable type. According to a further variant, instead of the continuous lateral wall 712 extending from the circular base 711, a discontinuous lateral wall or only a plurality of teeth orthogonally projecting from the circular base 711 and distributed around the perimeter edge of the circular base 711 can be provided, capable of preventing movements of the second portion 72 with respect to the first portion 71, when this is placed in contact with the circular base 711 of the first portion 71. Or vice versa, the second portion 72 can comprise a circular base and a continuous side wall extending from the circular base and delimiting therewith a housing seat and the first portion 71 is disk-shaped, with a diameter slightly smaller than that of the base circular of the second portion 72 and can therefore be inserted into the housing seat of the latter. The configurations of the connection elements 7 can be various, as long as they allow to obtain a removable coupling between their first portion 71 and their second portion 72.

As mentioned above, the removable coupling between the first portion 71 and the second portion 72 can advantageously also or only be of the magnetic type. In this regard, according to a particularly preferred embodiment of the invention illustrated in FIGS. 3a and 3b, the first portion 71 and the second portion 72 of a connection element 7 are respectively equipped with at least one magnet 73 (in the figures each portion is equipped with 4 circular magnets) allowing a magnetic attraction force to be developed between those two portions, which helps to keep them firmly bound to each other and yet makes their separation equally easy.

In FIGS. 3a and 3b, the circular magnets 73 are fixed in the first portion 71 and second portion 72, for example in respective seats specifically obtained, so that when the first portion 71 and the second portion 72 engage each other, the magnets can determine an attractive force therebetween. In the Figures, the magnets 73 are substantially arranged at the vertices of a square in the base 711 and in the disk 72, but the person skilled in the art will have no difficulty in understanding how their number and their arrangement can vary, provided that they allow developing between the first portion 71 and the second portion 72 a magnetic force sufficient to keep them bound in the normal conditions of use of the “smart” bag 1 of the invention.

Furthermore, the person skilled in the art will have no difficulty in understanding how, in the case wherein the coupling between the first portion 71 and the second portion 72 of a connection element 7 is of the magnetic type, it is not essential that the first portion 71 and the second portion 72 achieve a form fitting, since the magnetic force developed therebetween can be sufficient to prevent their detachment in the normal conditions of use of the “smart” bag 1 of the present invention, for their decoupling a detachment force greater than the magnetic attractive force generally developed between the first portion 71 and the second portion 72 during normal use of the “smart” bag 1 being required.

According to an aspect of the present invention, at least one connection element 7 of the plurality of connection elements can also include a reference third portion 74, configured to be applied to the item of clothing 4, at one external face thereof facing, in use, the surrounding environment. The reference third portion 74 can be directly connected to the item of clothing 4, for example glued, sewn or printed directly on the item of clothing 4, when the second portion 72 of the respective connection element is fixed to the item of clothing 4 at the respective internal face, as described above, or it can be configured to be applied to the item of clothing 4 directly connected to the second portion 72 of the connection element 7, for example by mechanical coupling, when, as described above, the second portion 72 of the connection element 7 is applied to the item of clothing 4, at the external face of that item of clothing 4. The reference third portion 74 is made of a transparent or semi-transparent flexible material, for example using an elastomeric material designed for applications requiring high elongation and high energy return, for example a material known by the trade name FormLabs elastic-50A produced by the US company Formlabs Inc., and a photopolymer resin developed by the applicant according to the teachings of international application WO2022/117544 A1, and on its external face it may show some relief areas, which constitute, for example, a number and/or a sign in Braille writing that identifies this connection element 7, so that even people with visual impairment can identify each connection element 7 and distinguish it from the others.

According to a preferred embodiment of the present invention, the connection elements 7 of the “smart” bag 1 are a plurality, for example four or more, optionally six, and each have the respective first portion 71 applied to the front face 20 of the hardware components pouch 2, if rectangular in shape, near one of its vertices. In the case represented in FIG. 4a, the connection elements are six, and the pouch 2 has the connection elements 7 applied to the front face 20 both at the four vertices and at a central region thereof, near the opposite edges of the bag 2. The respective second portions 72 are instead configured to be applied to the item of clothing 4, as described above, in corresponding positions, so that when the “smart” bag 1 of the present invention is applied to an item of clothing 4, each one of these second portions is located at a respective first portion 71 and can be coupled therewith.

However, a person skilled in the art will have no difficulty in understanding how the connection elements 7 of the “smart” bag 1 of the present invention can vary in number, provided that the bag 1 can remain in a fixed position with respect to the item of clothing 4, during its normal use.

Returning to the hardware components pouch 2, a plurality of emitting devices 8 is optionally but advantageously housed therein (see for example FIG. 1a), each emitting device 8 being operatively connected to the data control and processing unit 5 and configured for:
being activated, in response to a corresponding control signal s_{c}(t) sent by the data control and processing unit 5, and
emitting a corresponding emission signal sₑ(t).

According to a preferred embodiment of the invention, such emitting devices 8 comprise in particular vibrations emitting devices, indicated more particularly in FIG. 2 with reference number 80, one or more light radiations emitting devices, optionally LEDs, more particularly indicated in FIG. 2 with reference number 81, and at least one sound emitting device, more particularly indicated in FIG. 2 with reference number 82. These vibrations emitting devices 80 are comprised in the pocket 21 of the hardware components pouch 2 in a number equal to or less than the number of connection elements 7, and are advantageously housed in such pouch 2, each one at a first portion 71 of a respective connection element 7, at a minimum distance from the others not lower than 10 cm, optionally equal to 15 cm. This minimum distance is sufficient to avoid interferences with other vibrations emitting devices 80 and thus allows for a user of the “smart” bag 1 to clearly identify, while using the “smart” bag 1, which vibrations emitting device 80 is in operation at a given moment. Optionally, each light radiations emitting device 81 is also advantageously housed in the pocket 21 of the hardware components pouch 2, at a first portion 71 of a respective connection element 7. In this case, in the front face 20 of pouch 2, at each light radiations emitting device 81, corresponding through openings are obtained and also in the first portion 71 of the connection element 7 and in the respective second portion 72, as well as in the reference third portion 74, when present, corresponding through openings 75 are obtained (FIGS. 3a-3b), so that the light emitted by the light radiations emitting device 81 in the pouch 2 can be visible outside the pouch 2 and, when the bag is connected to an item of clothing 4, even externally thereto.

In the pocket 21 of the hardware components pouch 2 there is optionally also housed a plurality of switching devices 9, each switching device 9 being operatively connected to the data control and processing unit 5, and configured to be operated manually from a user and emit in response a corresponding switching signal s_{sw}(t) towards the data control and processing unit 5.

More particularly, the switching devices 9 comprised in the hardware components pouch 2 are pressure micro-switches, in a number equal to or less than the number of connection elements 7, and are advantageously housed in the pocket 21 of said pouch 2, each one at a first portion 71 of a respective connection element 7 so that, when a user exerts a pressure from the outside on the respective connection element 7, the switching device switches and transmits the respective switching signal s_{sw}(t) to the data control and processing unit 5.

The sound emitting device 82, unlike the other emitting devices (vibrations emitting devices 80 or light radiations emitting devices 81) and unlike the switching devices 9, can be housed in the hardware components pouch 2, in any position, not necessarily at a first portion 71 of a respective connection element 7.

For completeness, it is highlighted that all the hardware components described above, included in the pouch 2, are advantageously connected to the data control and processing unit 5, using conductive yarn, which does not weigh down the pouch 2, or using wires made of high quality copper, which ensure a high transmission speed of signals to and from the data control and processing unit 5. Clearly, in the hardware components pouch 2 a battery 10 is also included, for example a lithium battery, optionally of a rechargeable type through a special module optionally also contained in the pouch 2, the battery being connected in the same way to the data control and processing unit 5. The battery 10, as the person skilled in the art will easily understand, can be connected to the data control and processing unit 5 by means of an on-off switch.

The “smart” bag 1 described above can be connected to an item of clothing 4 of any size, as long as it is suitable for the purpose, i.e. an item of clothing 4 that remains sufficiently adherent to the user's body, when worn by him/her, so that the detectors 3 of the user's physiological parameters can correctly operate. Such an item of clothing 4 can be made, for example, of cotton or other breathable material and the independence of the “smart” bag 1 therefrom advantageously allows the inconvenience linked to the sizes available for “smart” clothing today available on the market to be resolved. By applying the “smart” bag 1 of the present invention to one's own item of clothing 4, each user will be able to have a “smart” garment of his/her own size. Not only that, since the “smart” bag 1 of the present invention can be easily removed from the item of clothing 4, by means of the mechanical and/or magnetic connection elements 7, the item of clothing 4 can be easily washed without running the minimal risk of damaging the electrical/electronic components.

As mentioned above, the “smart” bag 1 described above can be used to collect, store and exchange information to and from the surrounding environment, for example information relating to heart rate, breathing rate, body temperature, etc. of a user who is wearing it while carrying out a physical activity or at rest, or to collect, store and exchange information regarding the environment wherein the user is, such as environmental temperature, atmospheric humidity, position, etc.

Advantageously, the “smart” bag 1 of the present invention can also be used, when applied to an item of clothing 4 worn by a user, also to carry out cognitive exercises that help elderly or cognitively impaired people, both sighted and blind, maintaining short and long-term memory.

In this case, the “smart” bag 1 is configured to implement an operating method, which is the object of the present invention, which comprises according to a preferred embodiment, the following operational steps:
A. through the data control and processing unit 5, sending respective control signals s_{c}(t) to a plurality of emitting devices 8, according to a predefined delivery order, so that each emitting device 8, for example each vibrations emitting device 80 and each light radiations emitting device 81, when reached by the respective control signal s_{c}(t) emits a corresponding emission signal sₑ(t);
B. after a preset time interval has passed, detecting through the data control and processing unit 5 any switching signal s_{sw}(t) emitted by respective switches 9, in reply to a pressure exerted by the user on a respective connection element 7, and store the order of arrival of those switching signals s_{sw}(t);
C. through data control and processing unit 5:
comparing the preset delivery order of the control signals s_{c}(t) with the order of arrival of any received switching signals s_{sw}(t), and
storing the result of that comparison.

The operating method described above, after step C, optionally further comprises a step D of emitting a corresponding output signal sₒᵤₜ(t), toward I/O interface 6 of the hardware components pouch 2, for forwarding the result of the comparison to a remote device and its subsequent processing.

According to a further variant of the invention, the method comprises, before step A, also one step A₀ of emitting a control signal s_{c}(t) to the sound emitting device 82, if provided, which will emit a predefined sound emission signal sₑ(t), to acoustically warn the user about the start of transmission of control signals s_{c}(t) according to the predefined delivery order, by the data control and processing unit (5) to the corresponding emitting devices 8.

Similarly, according to a further variant of the invention, after step C, or D if provided, the method comprises a step E of emitting a control signal s_{c}(t) toward the sound emitting device 82, if present, which in reply will emit a sound emission signal sₑ(t), which depend on the result of the comparison carried out at step C, to acoustically warn the user of the end of the procedure and the match or not match of the predefined delivery order of the control signals s_{c}(t) with the order of arrival of the possibly received switching signals s_{sw}(t).

In the following, by way of example and not limitation, some implementing details are provided of an example of a “smart” bag 1 of the invention and the respective method of use, when the bag is connected to an item of clothing 4, specifically a t-shirt made of a breathable technical fabric.

According to this non-limiting example, the “smart” bag 1 of the invention comprises a cotton pouch 2, of rectangular shape, inside which are housed and connected, as described above:
the data control and processing unit 5, in particular a TTGO T-PCIE V 1.1 processor, marketed by the Chinese company Shenzhen Xin Yuan Electronic Technology Co., Ltd.;
an I/O interface 6, in particular a SIM7600E GSM/GPRS/GPS module, marketed by the Chinese company Shenzhen Xin Yuan Electronic Technology Co., Ltd., which allows high data transfer;
a heart rate and blood oxygenation detector 3, in particular model MAX30100, marketed by the Chinese company DollaTek;
a body temperature detector 3, in particular the model GY-906MLX90614 , marketed by the Chinese company RONGHAI Electronics Ltd.;
a temperature, humidity, environmental pressure and altitude detector 3, in particular model BME/BMP280, marketed by the German company AZ-Delivery Vertriebs GmbH;
an inertial unit 3 comprising a gyroscope and a 3-axis accelerometer, in particular model MPU-9250, marketed by the Chinese company RONGHAI Electronics Ltd.

In the hardware components pouch 2 of the “smart” bag 1 of the present invention there are also housed, as described above, six vibrations emitting devices 8, six (micro) switching devices 9 and one sound emitting device 8. The hardware components contained in pouch 2 are powered by a lithium battery, which develops at least 2000mAh, preferably 3000 mAh, via a charging module such as module TP4056, marketed by the German company AZ-Delivery Vertriebs GmbH, which includes a charge level indicator display that can be connected in any suitable way to the front face 20, facing outwards, so as to be visible when the battery 10 is turned on. To facilitate and improve the communication of the data control and processing unit 5 with the detectors 3, the emitting devices 8 and the switching devices, an expansion module is provided between that and the other components, for example the module MCP23017 IO, marketed by the Chinese company Waveshare Electronics. The connection elements 7 of the “smart” bag 1 are six, as described above and represented in FIGS. 3a-3c.

With reference to the method of operation of a “smart” bag 1 applied to the t-shirt made of breathable technical fabric, according to a first example, this can implement a short-term memory maintenance exercise, which requires by the data control and processing unit 5, the emission of predefined delivery sequences of control signals s_{c}(t) towards a number of vibration emitting devices 8 that is increased each time. Each control signal s_{c}(t) causes a vibration of the respective vibrations emitting device 80 for approximately 2 seconds. At the beginning and at the end of the delivery of the control signals s_{c}(t) according to the predefined delivery order, a sound is emitted via the respective sound emitting device 82 and any s_{sw}(t) switching signals, emitted by the micro-switches in response to the action of the user, who will have to replicate the delivery order of the previously transmitted control signals s_{c}(t), are detected by the data control and processing unit 5, during a subsequent defined time interval, optionally 10 seconds. After this time interval has passed, the sound emitting device 82 will emit a sound depending on whether or not the control signals delivery order corresponds to the receiving order of the detected switching signals.

A second exercise that can be implemented with the present invention is an exercise that promotes the maintenance of long-term memory. In this case, the data control and processing unit 5 emits control signals s_{c}(t) towards a number of vibration emitting devices 80 according to a predefined delivery order. Each control signal s_{c}(t) causes a vibration of the respective vibration emitting device 80 for approximately 2 seconds. At the beginning and at the end of transmission of control signals s_{c}(t) according to the predefined delivery order, a sound is emitted via the respective sound emitting device 82 and any s_{sw}(t) switching signal, emitted by the micro-switches in response to the action of the user, who will have to act on the switching devices 9, replicating the predefined delivery order of transmitted control signals s_{c}(t), is detected by the data control and processing unit 5, during a defined time interval, optionally 10 seconds. After this time interval has passed, the sound emitting device 82 emits a sound depending on whether or not the predefined delivery order of the control signals s_{c}(t) matches the receiving order of the detected switching signals. This procedure is repeated several times in a row so that the user can memorize the predefined delivery order. After some time, for example after about 30 minutes, following the emission of a sound by the sound emitting device 82, the user will have to replicate the predefined delivery order, acting on the (micro)switches 9, based on the memory of that order, which he/she will have kept in his/her memory.

Another exercise that can be implemented according to the invention requires that the data control and processing unit 5 emits control signals s_{c}(t) towards the respective vibration emitting devices 80, according to a predefined delivery order, wherein each control signal s_{c}(t) causes a vibration of the corresponding vibration emitting device 80 for about five seconds, during which the user will have to exert pressure on the corresponding connection element 7 and thus determine the switching of the corresponding switching device 9 and sending the switching signal s_{sw}(t) towards the data control and processing unit 5 for the aforementioned comparison.

In view of the above, the advantages offered by the present invention are evident.

First of all, thanks to the hardware components pouch 2 it is possible to make an item of clothing of any size “smart”, thus solving the inconvenience regarding the availability of sizes and the breathability of materials. Not only that, the hardware components of the “smart” bag 1 are not in direct contact with the user's body, when he/she is wearing the item of clothing connected to the “smart” bag 1, providing a more comfortable use. The hardware components pouch 2, which can be easily separated from item of clothing 4, allows the latter to be washed without running the slightest risk of damaging its contents.

In the foregoing, the preferred embodiments have been described and variations of the present invention have been suggested, but it is to be understood that those skilled in the art will be able to make modifications and changes without departing from the corresponding scope of protection, as defined by the attached claims.

1. A smart bag configured to be connected to an item of clothing, said bag comprising:
one hardware components pouch, having a front face and a rear face opposite to the front face, between the front face and the rear face at least one pocket being delimited, which pocket is in communication with the outside of pouch through one opening and, inside the pocket being housed:
a plurality of detectors, each detector being configured to detect one or more environmental parameters about the environment around the bag or one or more physiological parameters of a user, when the user is wearing an item of clothing connected to said bag, in reply to a respective activation signal sₐ(t) received in input, and output a corresponding detection signal sₛ(t);
a data control and processing unit, operatively connected to the plurality of detectors and configured to send said activation signal sₐ(t) to each one of said detectors and receive, in reply, the corresponding detection signal sₛ(t); and
a plurality of connection elements, configured far removably connecting the hardware components pouch to said item of clothing;
wherein each connection element of the plurality of connection elements comprises:
at least one first portion, configured far being fixed on the same face of the hardware components pouch, in a permanent or removable way; and at least one second portion, configured, to be removably engaged with the respective first portion, on one side, and to be applied to the item of clothing, on the other side.

2. A smart bag according to claim 1, wherein the at least one second portion is configured to be applied to the item of clothing permanently or removably, the inner face of said item of clothing facing the body of the user, when the user is wearing said item of clothing.

3. A smart bag according to claim 1, wherein the at least one second portion is configured to be applied to the item of clothing, in a permanent or removable way, the outer face of said item of clothing, facing the environment around the user, when the user is wearing said item of clothing.

4. A smart bag according to claim 1, wherein the removable engagement between the at least one first portion and the at least one second portion, is a mechanical and/or magnetic engagement.

5. A smart bag according to claim 4, wherein in the at least one first portion and the at least one second portion, at least one magnet is housed, when the engagement between the first portion and the second portion is of magnetic type.

6. A smart bag according to claim 1, wherein at least one connection element of the plurality of connection elements also comprises a reference third portion, configured to be directly engaged with the item of clothing, when the at least one second portion of the respective connection element is applied to the item of clothing at the inner face thereof, or configured to be applied to the item of clothing directly engaged with the at least one second portion of the connection element, when the at least one second portion of the connection element is applied to the item of clothing at the outer face of said item of clothing, the outer face of the reference third portion optionally having some raised areas, farming a number and/or a sign in braille writing, which identifies said connection element.

7. A smart bag according to claim 1, comprising a plurality of emitting devices housed inside the pouch, each emitting device being arranged at a first portion of a respective connection element, and being operatively connected to the data control and processing unit and being configured for:
being activated, in reply to a corresponding control signal s_{c}(t) sent by the data control and processing unit, and
emitting a corresponding emission signal sₑ(t).

8. A smart bag according to claim 1, comprising a plurality of switching devices housed inside the pocket, each switching device being arranged at a first portion of a respective connection element, and being operatively connected to the data control and processing unit and configured for being manually operated by a user, through a pressure exerted at least on the respective second portion, and emitting in reply a corresponding switching signal s_{sw}(t) toward the data control and processing unit.

9. A smart bag according to claim 1, wherein the hardware components pouch (2) is made of a breathable fabric and/or wherein each detector, each emitting device and each switching device is operatively connected to the data control and processing unit through a conductive yarn.

10. A smart bag according to claim 1, comprising at least one input/output I/O interface, housed inside the pouch and operatively connected to the data control and processing unit, and configured for allowing one output signal sₒᵤₜ(t) transmission toward the outside to a remote device, said signal being outputted by the data control and processing unit, and far allowing receiving in input and transmitting to the data control and processing unit one or more input signals sᵢₙ(t), transmitted by the remote device, the remote device optionally being a portable phone, tablet, pc, a remote server or other suitable processing unit.

11. The smart bag according to claim 1, wherein the smart bag is connected to an item of clothing.

12. A method of using a smart bag (1), when the bag (1) is applied to an item of clothing, the method comprising the following operational steps:
A. through a data control and processing unit, sending respective control signals s_{c}(t) to a plurality of emitting devices, according to a predefined delivery order, so that each emitting device, reached by the respective control signal s_{c}(t) emits one emission signal sₑ(t);
B. after a preset time interval, detect through the data control and processing unit any switching signal s_{sw}(t) emitted by respective switching devices, in reply to a pressure exerted by the user on the respective connection element, and storing the order of arrival of said switching signals s_{sw}(t);
C. through the data control and processing unit: comparing the predefined delivery order of the control signals s_{c}(t) with the order of arrival of any received switching signals s_{sw}(t), and storing the result of said comparison.

13. The method according to claim 12, further comprising a step D of emitting, by the data control and processing unit toward I/O interface, one output signal sₒᵤₜ(t) based on the result of said comparison carried out at step C, for forwarding by said I/O interface to the remote device and its subsequent processing.

14. The method according to claim 12, further comprising, before step A, also one step A₀ of emitting a control signal s_{c}(t) toward a sound emitting device, if provided, that causes the emission of a predefined sound emission signal sₑ(t), to acoustically warn about the start of transmission of control signal s_{c}(t) according to the predefined delivery order, by the data control and processing unit, and/or comprising after step C, or D is provided, one step E of emitting a control signal s_{c}(t) toward the sound emitting device (82), if provided, that causes the emission of a sound emission signal sₑ(t), the sound of which depends of the result of the comparison carried out at step C, to acoustically warn the user of the match or not match of the predefined delivery order of control signals s_{c}(t) with the order of arrival of the possibly received switching signals s_{sw}(t).
